(19) United States
(12) Patent Application Publication
(10) Pub. No.: US 2023/0277356 A1
(43) Pub. Date: Sep. 7, 2023

(54) ORTHOSIS SYSTEM

(71) Applicant: TRITON SYSTEMS, INC., Chelmsford, MA (US)

(72) Inventors: Marmaduke J. Loke, Lowell, MA (US); Marya H. Schnedeker, Cambridge, MA (US); Michael J. Guarino, Wakefield, MA (US); William M. Lindsey, Lowell, MA (US); James F. Saunders, Lowell, MA (US); George Santiago, Marlborough, MA (US); Brian E. Moyer, Nashua, NH (US)

(21) Appl. No.: 18/176,966

(22) Filed: Mar. 1, 2023

Related U.S. Application Data
(60) Provisional application No. 63/315,424, filed on Mar. 1, 2022.

(51) Int. Cl. A61F 5/058

(52) U.S. Cl. CPC A61F 5/0585; A61F 5/05825

(57) ABSTRACT

Disclosed is an orthosis device particularly well-suited for use in the field. The orthosis system includes a tibial cuff, at least one strut, an optional off-loading cuff, and a foot plate. The struts connect the tibial cuff to the footplate. Optional couplers may be used to facilitate these connections and may be in the form of quick disconnects or cam locks. The footplate may further spring plate having energy return properties to aid mobility. The systems may be modular, is compact, and easily stowed and carried.

### CROSS-REFERENCE TO RELATED APPLICATIONS

This application claims benefit of priority to U.S. Provisional Patent application No. 63/315,424 entitled ORTHOSIS SYSTEM filed on Mar. 1, 2022, the entirety of which is hereby incorporated by reference.

### RELATED APPLICATIONS

This application is related U.S. Provisional Patent Application No. 63/315,404 entitled Garment-Based Splint System, and its contemporaneously filed non-provisional patent application, each of which is hereby incorporated by reference in its entirety.

### GOVERNMENT SPONSORSHIP

This invention was made with government support under contract nos. W81XWH-21-C-0062 and W81XWH2OP0054 awarded by the US Army Medical Research Acquisition Activity (USAMRAA). The government has certain rights in the invention.

### BACKGROUND

The 2018 National Defense Strategy identified “Forward force maneuver and posture resilience” as a key capability that must be modernized to “solidify our competitive advantage.”

Currently available solutions are less than desirable. Powered exoskeletons are bulky, heavy, expensive, and require complex adjustment to fit an injured person's body. Formable splints and braces are not able to fully immobilize an injured limb, especially during subsequent movement, which increases the risk of re-injury. Vacuum splints are very heavy and render the casualty non-ambulatory. Immobilizing walking boots are large and heavy. Simple braces and wraps can effectively treat minor sprains but are not well suited for more severe injuries. Air-casts require additional hardware (a pump) to apply and are not designed for use in austere environments. Plaster and or fiberglass casts require advanced skills to apply, copious pack volume, and significantly more time to apply and set than this garment. Pinned fixators require advanced training and specialized tools to apply, at times including imaging equipment, and necessitate puncturing the skin which can lead to additional complications.

Thus, new and improved injury support systems are needed that:

Improve warfighter resilience and survivability, in conjunction with orthotic elements, allowing injured warfighters to return to mission safely and effectively despite having sustained lower-limb injuries that would otherwise diminish their capabilities by reducing undesirable motion of an injured limb near the ankle.

Improve medic effectiveness by reducing pack footprint, required application time, training, and needed skill compared to the current standard of care.

Improve unit effectiveness by promoting buddy-care (or possibly even self-care) to reduce task burdens on medics allowing them to respond to injuries requiring expert care.

### SUMMARY

The disclosure describes an orthosis device.

Some embodiments provide an orthosis device comprising a first support structure having a first connector; a second support structure having a second connector, one or more extensible struts, adapted to be extensible to a desired length, and to be connected at opposite ends to the first connector and the second connector, whereby the extensible strut distributes a load between the first support structure and the second support structure.

In some embodiments, the first support structure is a footplate, and the second support structure is a tibial cuff. In some embodiments, an off-loading cuff suspension which wraps around the injured limb and suspends it from the tibial cuff, reducing or eliminating the load transferred through the injury site may also be used.

In some embodiments the two or more extensible struts are telescoping struts.

Some embodiments provide a kit comprising an orthosis device described herein; and a splint system. In some embodiments, the splint system is a garment-based splint system. In others, it is a foam-based splint system.

### BRIEF DESCRIPTION OF THE DRAWINGS

Aspects, features, benefits, and advantages of the embodiments described herein will be apparent with regard to the following description, appended claims, and accompanying drawings where:

FIG. 1 depicts an embodiment disclosed herein, including the optional off-loading cuff.

FIG. 2 is a depiction of an orthosis as described herein and a similar view in use.

FIG. 3 is a top planar rendering of a foot plate in accordance with some embodiments disclosed herein.

FIG. 4 is a series of photos showing different footplate geometries and connectors.

FIG. 5 is a cross-sectional view of a foot plate assembly (several details are missing for clarity).

FIG. 6 shows three suitable connector systems.

FIG. 7 is a rendering of a tibial cuff in accordance with some embodiments.

FIG. 8 depicts the system disassembled and stored for transport.

FIG. 9 is a series of photos depicting a tibial cuff in accordance with some embodiments.

This disclosure is not limited to the particular systems, devices and methods described, as these may vary. The terminology used in the description is for the purpose of describing the particular versions or embodiments only, and is not intended to limit the scope.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also understood that this disclosure is not limited to particular compositions, methods, apparatus, and articles, as these may vary. It is also to be understood that the terminology used herein is for the purpose of describing particular embodiments only, and is not intended to be limiting.

Disclosed herein is an orthosis system (e.g., splint system) that allows for a quick return to mobility with minimal or no support needed from another person. This is particularly useful to sustain combat power and readiness in military applications by rapidly returning injured warfighters to duty in operational environments. This will be increasingly important in future conflicts where uncontested air superiority cannot be guaranteed. Further, it can continue to augment the warfighter's capabilities up the echelons of care until definitive treatment is available. The system is compact in size while stored, often smaller than splinting supplies currently carried in a medic bag, while offering significantly better performance. The feasibility of a prototype splint system as disclosed herein has been demonstrated to address the number one injury requiring MEDEVAC.

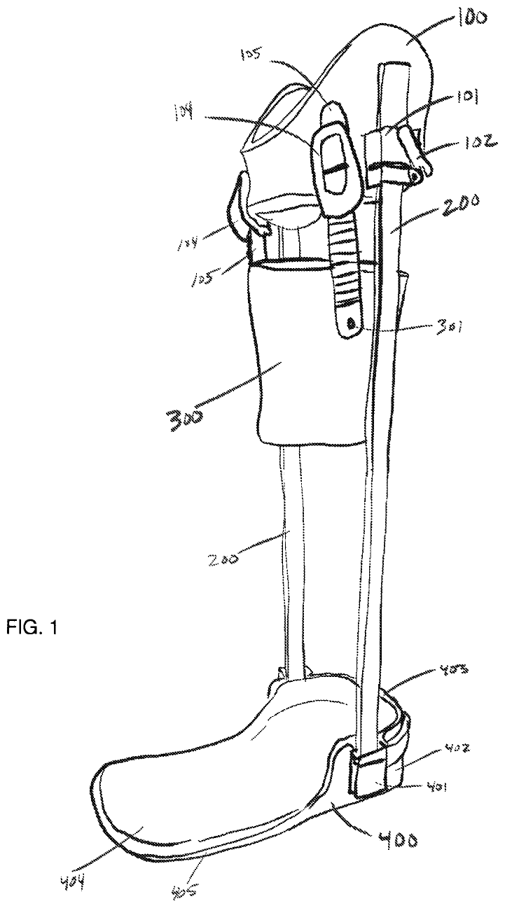

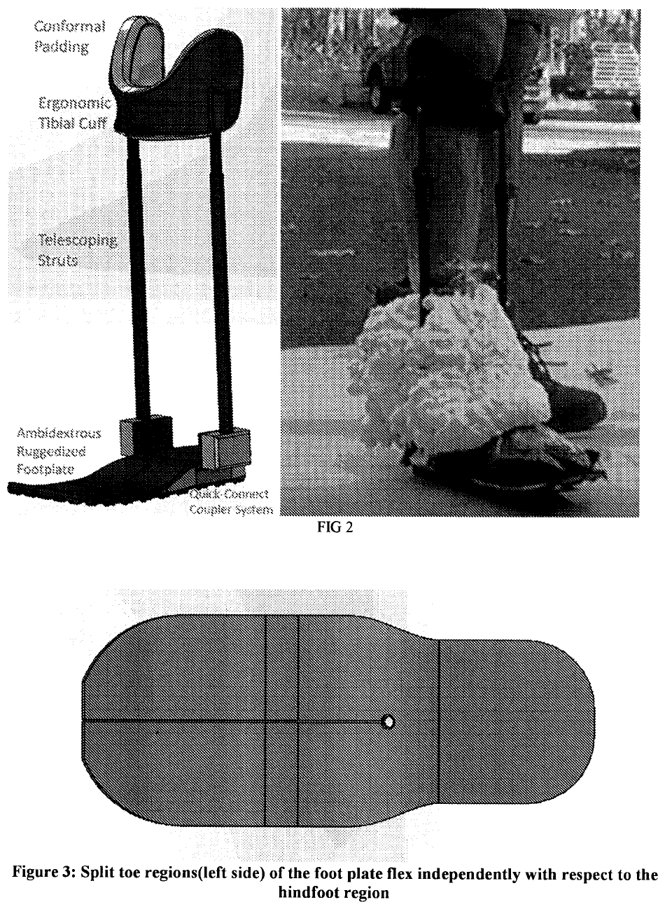

FIG. 1 illustrates an exemplary embodiment of the orthosis system disclosed herein. The orthosis system includes a tibial cuff 100, two struts 200, an optional off-loading cuff 300, and a foot plate 400. The struts 200 connect the tibial cuff 100 to the footplate 400. Optional couplers (proximal coupler 101, distal coupler 401) may be used to facilitate these connections and may be in the form of quick disconnects or cam locks 102. The footplate may further comprise a distal coupler 401, a heel pad 402, a heel cup 402, a toe pad 404, and a spring plate 405. The spring plate 405 in some embodiments is design as an energy return device to aid mobility. The optional off-loading cuff 300 is suspended from the tibial cuff 100 by an offloading strap 105 which may include an offloading strap buckle 104 which together may allow for height adjustment of the off-loading cuff 300. The offloading strap 105 may be connect to the tibial cuff and the off-loading cuff by any suitable means, such as a rivet (see e.g. 301).

In some embodiments, the orthosis system comprises a weight-bearing support structure that strengthens the splint and allows the wearer to maintain mobility, including remaining ambulatory while reducing the load on the injury, including telescoping struts between two support structures that work to divert load bearing around and away from the injured area. In some embodiments, an off-loading cuff may be used to further support the injured area.

In embodiments designed to address lower leg, foot, or ankle injury, a durable foot plate is provided. It is affixed to the injured foot to reduce roll-over and facilitates maintaining proper gait despite the immobilization of the ankle joint.

Optionally, a splint or casting system may be used. For example, casting systems such as described in U.S. Pat. Nos. 9,427,489 and 10,350,103, each of which is incorporated herein by reference, may be used in conjunction with this system. In some embodiments, a garment-based splint system such as described in the U.S. Provisional Patent Application entitled Garment-Based Splint System, which is being contemporaneously filed herewith and is hereby incorporated by reference, may also be used.

A filler component to aid in stabilization, such as a foam (e.g., a fast-curing rigid polyurethane foam) that provides near-instant immobilization, a liquid, gel, gas, non-air gas, etc. may be used alone or with such splinting applications in conjunction with the orthosis described herein.

The disclosed design does not suffer from key limitations present in competing treatment options. Powered exoskeletons are bulky, heavy, expensive, and require complex adjustment to fit an injured person's body. Formable splints and braces are not able to fully immobilize an injured limb, especially during subsequent movement, which increases the risk of re-injury and, although it is possible to create weight-bearing splints with currently available formable products, doing so often requires training/skills, a time-consuming process to administer, and significant medical bag volume. Vacuum splints are very heavy and render the casualty non-ambulatory. Immobilizing walking boots are large and heavy and, although designed to prevent ankle motion, do not offload the injured limb and are not designed for rough terrain. Simple braces and wraps can effectively treat minor sprains but are not well suited for more severe injuries especially because they do not provide any limb off-loading. Air-casts require additional hardware (a pump) to apply and are not intended for load-bearing applications. Plaster and or fiberglass casts require training to apply, copious pack volume, and significantly more time to apply and set than the system described herein and make walking much more difficult once applied. Pinned fixators require advanced training and specialized tools to apply, at times including imaging equipment, and necessitate puncturing the skin which can lead to additional complications.

The solution provided herein is compact and light, assemble-able in minimal time with minimal skill using no tools or power source, easily adjustable to fit both left and right lower limbs for a wide range of individuals (5th percentile female to 95th percentile male), or easily adapted to specific sizes such as petite or extra-large, able to limit or prevent undesirable motion of the injured limb even while loaded during gait, capable of transferring body weight through the orthosis to a point proximal to the injury, designed to promote more efficient gait through a novel energy capture and return mechanism, etc.

In some embodiments, the system is modular and parts may be used from system to system to create different arrangements, sizes, and combinations to address a variety of injuries and injured parts.

Generally, the orthosis, which is adapted to work for either left or right leg injuries, includes a foot bed, a tibial cuff, extensible struts, and quick disconnects to the extensible struts to connect the extensible struts to either or both the foot bed and tibial cuff. Optional splint or casting systems may be used in conjunction with this orthosis.

The Footbed

The footbed provides a platform that flexes with respect to the hindfoot during the terminal stance phase and then returns to its original configuration. In some instances, this is adapted to provide propulsive energy return, as the orthosis transitions to the swing phase of gait, thus making up for the inability to use the calf muscle.

In some embodiments, the footbed is provided with separated lateral and medial forefoot platforms (FIG. 3) These forefront platforms flex with respect to the hindfoot during the terminal stance phase and then return to their original configuration, providing propulsive energy return, as the orthosis transitions to the swing phase of gait. In addition, flexing of the independent forefoot regions in response to rough terrain underfoot provides energy storage and, by flexing differentially to higher and lower obstacles, provides a more stable platform to help an injured warfighter maintain balance while reducing frontal plane loads that would otherwise affect more proximal joints. The use of the terms lateral and medial are for convention only. It will be clear to those of ordinary skill in the art that since the device can be used on either leg, that the nomenclature refers to opposite sides of the orthosis.

In embodiments that provide propulsive energy, the foot bed may be made from materials which are strong, yet flexible, and capable of storing and returning energy as described.

The footbed includes a cushioned heel region and boot tread designed to match the characteristics of standard issue combat boots while providing energy dissipation during heel strike. This cushion absorbs shock and replaces plantarflexion in early stance phase to provide a smooth transition to mid-stance. The footbed may include additional padding and securing straps to maintain device placement or to assist with the application process. In some embodiments, the foot plate can be used both with and without additional footwear. The patient can wear the orthosis in lieu of other foot coverings, or it can accommodate a boot/shoe.

The hindfoot area of the foot bed is adapted to be connected to one end of each extensible strut. This may be done by any suitable means, including, but not limited to, a socket, or via integrated quick-connectors. Some embodiments may use more than two connecting struts. Other embodiments might use a single strut. Although quick connectors are discussed herein, other connecting structural components may be used alone or in conjunction with quick connectors.

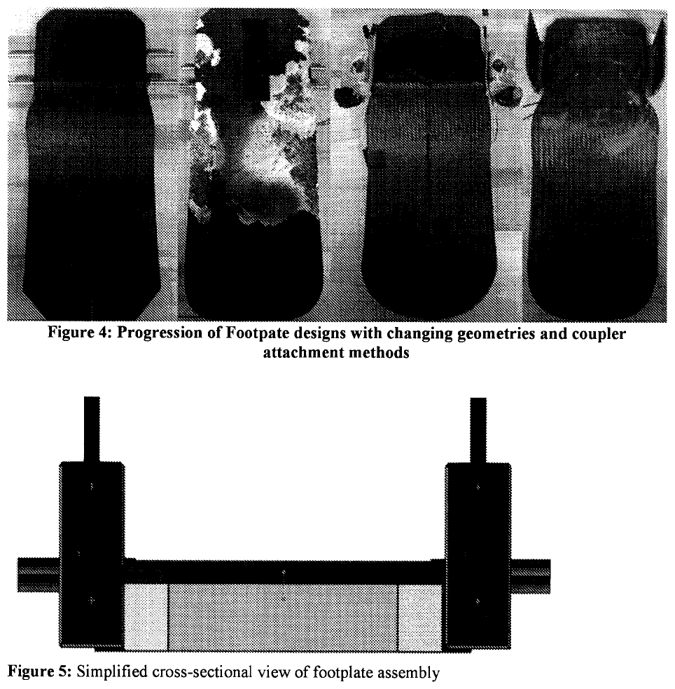

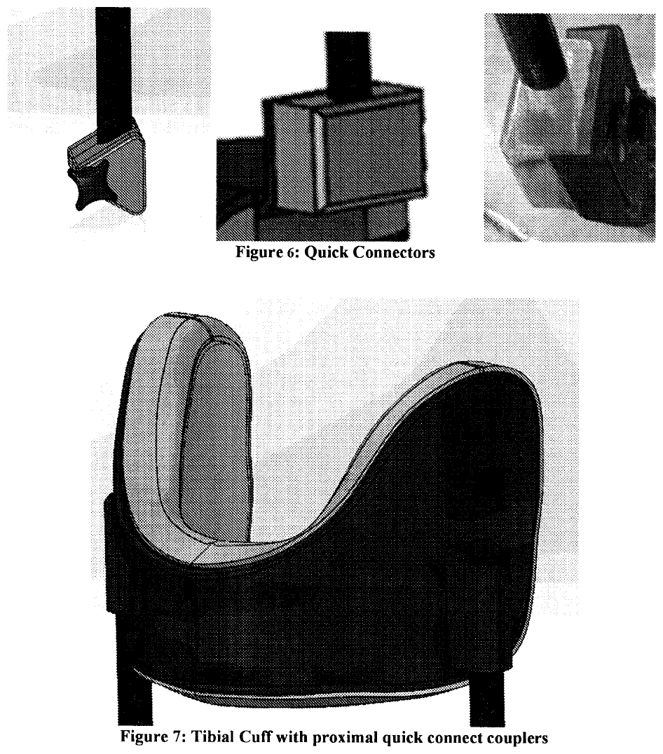

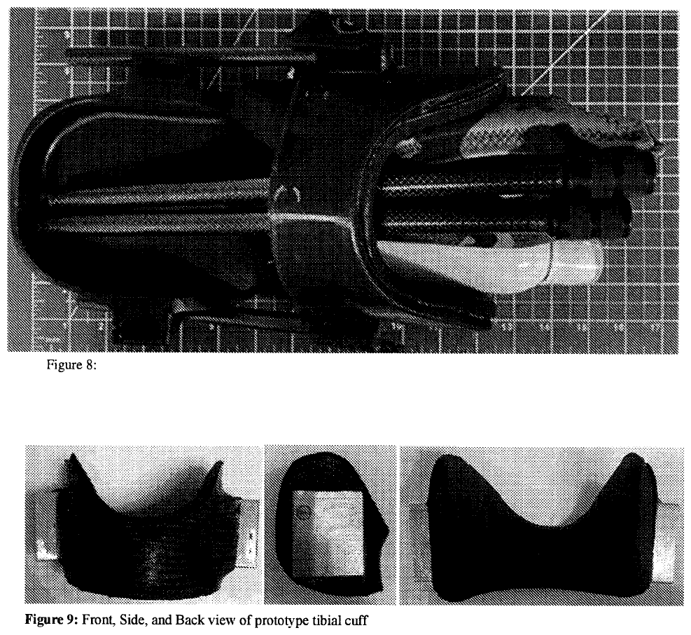

The footplate can take various shapes and sizes as depicted in FIG. 4, which also shows various connector designs.

The cross-sectional image of the prototype model in FIG. 5 shows an iteration of the design with one structural strut, partially nested within the footplate. The cross-sectional image simplifies the overall footplate assembly to isolate the structural support struts. Although the top portion of the footplate is shown (black material), there is no underside support shown, neglecting any additional weight management. By calculating the forces acting, and subsequent stresses, within the struts using this simplified model, subsequent shear forces and potential deflection that would be displayed by struts of variant diameters and material composition were modeled to evaluate various designs.

Tibial Cuff

The tibial cuff (FIG. 7) provides the connection between the load transfer struts and the shank above the point of injury and has been designed to work for a range of individuals from 5th percentile female to 95th percentile male. Special attention has been paid to prevent painful point loads between the cuff and human tissue for both right and left-side injuries.

The cuff is designed and fabrication to the geometry of both left and right limbs. Device symmetry allows for a reduced medical pack volume demanded by the deployable system, avoiding the need for both a left and right device configuration. A rigid cuff also offers solid alignment performance, maintaining the alignment of the knee and foot to prevent any rotational instability in the damaged ankle; flexible alternatives are not predicted to perform as well and may not offer an identical level of device trust. Some embodiments provide an adjustable cuff that can be adjusted to accommodate larger or smaller limbs or for comfort.

Although rigid materials (with comfort padding) are contemplated, the use of a more malleable material aimed at delivering enhanced levels of adjustability and user comfort may be employed. A less rigid structure for this component will also allow for unassembled compression and reduced space demanded within the medic pack. This design, among other things, accounts for proper transverse alignment of the limb while optimizing for size, weight, cost, and manufacturability.

The tibial cuff has proximal flares extending on either side of the knee to facilitate transverse alignment of left/right limb to prevent further injury. That is, the tibial cuff helps maintain knee and foot alignment. The cuff also provides controlled, anterior resistance to load the footplate during stance phase. The cuff allows weight to be unloaded from the ankle joint to avoid further injury and to reduce pain from loading through that joint and accounts for mirrored pressure sensitive areas such as fibula head, lateral tibial condyle, peroneal and infrapatellar saphenous.

The tibial cuff is designed to partially encircle the leg, under the knee, with proximal flares that extend to the side of the knee. The lower portion of the tibial cuff is provided with a coupler on each side to couplet to an extensible strut, which is in turn connected to the foot bed. The coupler may be a quick disconnect.

The optional off-loading cuff may be removeable. The purpose of the off-loading cuff is to suspend the injured part from the tibial cuff. To accomplish this, the off-loading cuff is attached to the tibial cuff and suspended therefrom such that at least a portion of the weight of the limb below is support by the tibial cuff via the off-loading cuff, thereby putting less weight and pressure on the injured leg/ankle/foot. The weight is instead carried by the orthosis, which transfers the weight via the one or more struts to the footplate.

Extensible Struts

An extensible strut is provided on either side of the orthosis, corresponding to lateral and medial positions. Each strut extends from the tibial cuff to the footplate. Each extensible strut is secured, in some instances by a quick disconnect, to the footbed and the tibial cuff. These struts function to transfer the load off the injured area. That is, the load is transferred from the footbed to the tibial cuff, bypassing an injured ankle or lower leg. In some embodiments, the extensible struts are telescopic. Other extensible mechanisms may be used. The length adjustment accommodates a wide range of leg lengths. The cross section of the strut used can vary based on application. In some embodiments, one or more of the struts may pass through at the cuff to extend further up the leg with a locking mechanism securing the cuff to the strut at a desired point to set the distance between the footplate and the cuff. A longer strut might enable splinting across an injured knee through the cuff to a more proximal mounting point on the thigh.

The extensible struts are chosen to balance strength, weight and pack volume. The orthosis system disclosed herein can outperform splinting systems currently leveraged by military medics.

The struts may be solid or hollow, depending on material properties. Any cross-section geometry may be used, including but not limited to, rectangular or cylindrical. Unlike most solid ankle devices, such as the IDEO, which store their dynamic energy in the strut causing a degree of ankle motion, the orthosis disclosed herein relies on a more rigid strut, storing energy within the footplate instead. This is aimed at protecting a damaged ankle of the end user. A hollow strut affords the possibility of nesting components within one another, contributing to a reduced, unassembled volume. Although asymmetrical, two struts of different diameter and wall thickness may be used to match member stiffnesses while providing the ability to nest one piece within the diameter of a larger piece in an effort to create another space-saving advantage.

These lightweight and stiff struts are designed for easy assembly by individuals with no special training or advanced skills and allow the device to accommodate from 5th percentile female to 95th percentile male users. While a standard device can cover this wide range, the concepts used herein are readily adaptable to accommodate those outside that range or to be more closely tailored to a specific range.

Quick Connectors

The quick connectors (Figure) provide a high-strength attachment method that requires no additional tools or special skill/training to secure the footplate or the tibial cuff to the struts. Any suitable quick connector may be used. The connectors will be designed to work effectively under different environmental conditions and the tightening mechanism will prevent fouling caused by debris. Some examples are provided below.

Embedded Footplate Struts

An innovative assembly method includes mating struts to couplers which were grafted between layers of the composite laminate to create precision, toolless quick disconnects.

Press Fit Couplers

A toolless method for joining struts to a footplate or tibial cuff via a solid coupler which tightly mates with the inner or outer surface of struts or with a strut end cap when pressed in by a user. Coupler retention is maintained by friction, often increased through induced strut flexure in the frontal plane adjusted by intended misalignment of distal and proximal couplers.

Cam Lock Couplers

A toolless method for joining struts to a footplate or tibial cuff via a solid coupler which loosely mates with the inner or outer surface of struts or with a strut end cap. Coupler retention is maintained via a section of the coupler tightening on the strut or cap via a cam lock or tightening screw.

Screw in Couplers

A toolless method for joining struts to a footplate or tibial cuff via a solid coupler with a tapped hole which mates with a threaded outer surface of a strut end cap.

Anterior Strut

A secondary strut connected from the mid-foot or fore-foot section of the footplate to the anterior tibial cuff to be used in combination with a primary strut(s) configuration, used to transfer force from the footplate to the proximal leg. This creates a very stiff coupling between the tibial cuff and footplate, preventing ankle-flexion. Alternatively, a shell style (thin walled) wrap around layer may be used to provided a more organic surface to connect the footbed to the cuff.

Splint or Casting Systems.

The orthosis described herein is well suited for use in conjunction with a splint or casting system to further stabilize the injured leg. As noted above, casting systems such as described in U.S. Pat. Nos. 9,427,489 and 10,350,103, each of which is incorporated herein by reference, may be used in conjunction with this system. In some embodiments, a garment-based splint system such as described in the U.S. Provisional Patent Application entitled Garment-Based Splint System, which is being contemporaneously filed herewith and is hereby incorporated by reference, may also be used.

Kits

In some embodiments, a kit, incorporating the various parts of the orthosis system described herein may be provided. In some embodiments, the kit further comprises a splint or casting system. In some embodiments, the splint system is a garment-based splint system such as described in the contemporaneously filed U.S. provisional patent application referenced above.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

This disclosure discusses the orthosis with respect to use to stabilize the lower leg, but similar concepts can be used with other areas and joints, such as but not limited to knee, elbow, wrist, neck, shoulder, back, hand, foot, long bones, etc.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of or “consist of the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As used herein, the term “about” means plus or minus 10% of the numerical value of the number with which it is being used. Therefore, about 50% means in the range of 45% 55%.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. An orthosis system comprising:
a tibial cuff;
one or more struts;
and a foot plate 400;
wherein the struts connect the tibial cuff to the footplate.

2. The orthosis system of claim 1, further comprising an off-loading cuff which is suspended from the tibial cuff.

3. An orthosis device comprising:
a first support structure having a first connector;
a second support structure having a second connector,
two or more extensible struts, adapted to be extensible to a desired length, and to be connected at opposite ends to the first connector and the second connector, whereby the extensible strut distributes a load between the first support structure and the second support structure.

4. The orthosis device of claim 3, wherein
the first support structure is a footplate, and
the second support structure is a tibial cuff.

5. The orthosis device of claim 4, wherein the two or more extensible struts are telescoping struts.

6. A kit comprising:
an orthosis device in accordance with claim 1; and
a splint system.

7. The kit of claim 6, wherein the splint system is a garment-based splint system.

8. The kit of claim 6 further comprising a foam-based splint system.
